(19) United States
(12) Patent Application Publication
(10) Pub. No.: US 2014/0107780 A1
(43) Pub. Date: Apr. 17, 2014

(54) Voice Prosthesis

(71) Applicant: Robert G. Perry, Indianapolis, IN (US)

(72) Inventor: Robert G. Perry, Indianapolis, IN (US)

(21) Appl. No.: 13/653,484

(22) Filed: Oct. 17, 2012

(51) Int. Cl. A61F 2/20

(52) U.S. Cl. 623/9

(57) ABSTRACT

A voice prosthesis device is disclosed that includes an improved fluid seal. The device includes a hollow cylindrical tube having flexible retainer flanges integrally formed over the open ends of the tube. A low pressure check valve is disposed within the tube for allowing air to pass from the trachea to the esophagus of the user when the user desires to speak. The external surface of the cylindrical tube includes one or more annular fluid seal projections or ribs that improve the fluid seal when the device is disposed in a tracheal-esophageal stoma of a user. The voice prosthesis device is preferably fabricated from semi-rigid resilient and flexible materials.

### FIELD OF THE INVENTION

This invention relates in general to prosthetic medical devices and more particularly to voice prosthesis devices.

### BACKGROUND OF THE INVENTION

I, Bob Perry, inventor of the present invention, underwent a laryngectomy operation. As a result of the laryngectomy procedure, a tracheal-esophageal puncture was performed and I received a voice prosthesis installed in the resulting stoma. I also wear an endostomal tracheostoma button and accompanying tracheostoma valve for breathing purposes. I have experienced annoying difficulties with regard to fluid leakage between my trachea and esophagus past the voice prosthesis device. When my voice prosthesis has a fluid leak, food and fluids that I consume pass from my esophagus and into my trachea or windpipe with irritating results. Such leaks occur as a result of ineffective seals in the voice prosthesis device. When leaks occur, I am forced to remove the tracheostoma valve and tracheostoma button and cough to expel fluid or food from my windpipe and lungs. Coughing is, at the least, quite a process to accomplish as well as publicly embarrassing for a laryngectomy patient.

The typical remedy for fluid leaks past the voice prosthesis device is the installation of a new voice prosthesis, normally installed by medical professionals who provide my care. Unfortunately, the new voice prosthesis does not have a reliable fluid seal design and fluid leaks occur rather soon after a replacement voice prosthesis is installed. It occurred to me that a smooth cylindrical surface with circular flanges at the ends thereof is not a very effective fluid seal design given the environment where my voice prosthesis is placed and the fluid seal issues at that location, including the depth or length of my trachea-esophagus puncture and the size or geometry of the puncture.

What is needed is a better voice prosthesis device with an improved fluid seal design. I have endeavored to improve the design of an existing voice prosthesis devices, in particular, the fluid seal between the trachea and esophagus, by altering the external surfaces of an existing voice prosthesis. The following invention description details my design improvement for such voice prosthesis devices.

### SUMMARY OF THE INVENTION

A voice prosthesis device, according to one aspect of the present invention, comprises a hollow cylindrical tube having an external lateral surface, a first open end, a second open end, and at least one annular protrusion extending radially outward a fixed distance from the external lateral surface of the hollow cylindrical tube, and wherein the radius of the tube is sized for insertion into a stoma connecting a users trachea and esophagus, low pressure check valve means for allowing air to pass through the cylindrical tube when air pressure on one side of the cylindrical tube exceeds a predetermined level, the valve means being disposed within the cylindrical tube, a first retainer flange attached over the first open end of the hollow cylindrical tube, the first retainer flange having a disk-like shape and an aperture aligned with the first open end, and a second retainer flange attached over the second end of the hollow cylindrical tube, the second retainer flange having a disk-like shape and an aperture aligned with the second open end.

One object of the present invention is to provide an improved voice prosthesis device.

Another object of the present invention is to improve the fluid seals for a voice prosthesis device disposed in a stoma communicating between the trachea and the esophagus.

Yet another object of the present invention is to extend the life expectancy of voice prosthesis devices after installation thereof.

These and other objects of the present invention will become more apparent from the following description of the preferred embodiments.

### BRIEF DESCRIPTION OF THE DRAWINGS

FIG. 1 is a partial cross-sectional view of a patient's throat depicting a voice prosthesis of the prior art installed.

FIG. 2 is a side view of a voice prosthesis according to one aspect of the present invention depicted in a cross-section of a patient's throat.

FIG. 3 is a front view of the voice prosthesis of FIG. 2.

FIG. 4 is a side view of a voice prosthesis according to another aspect of the present invention shown in a cross-section of a patient's throat.

### DESCRIPTION OF THE PREFERRED EMBODIMENTS

For the purposes of promoting an understanding of the principles of the invention, reference will now be made to the embodiments illustrated in the drawings and specific language will be used to describe the same. It will nevertheless be understood that no limitation of the scope of the invention is thereby intended, such alterations and further modifications in the illustrated devices, and such further applications of the principles of the invention as illustrated therein being contemplated as would normally occur to one skilled in the art to which the invention relates.

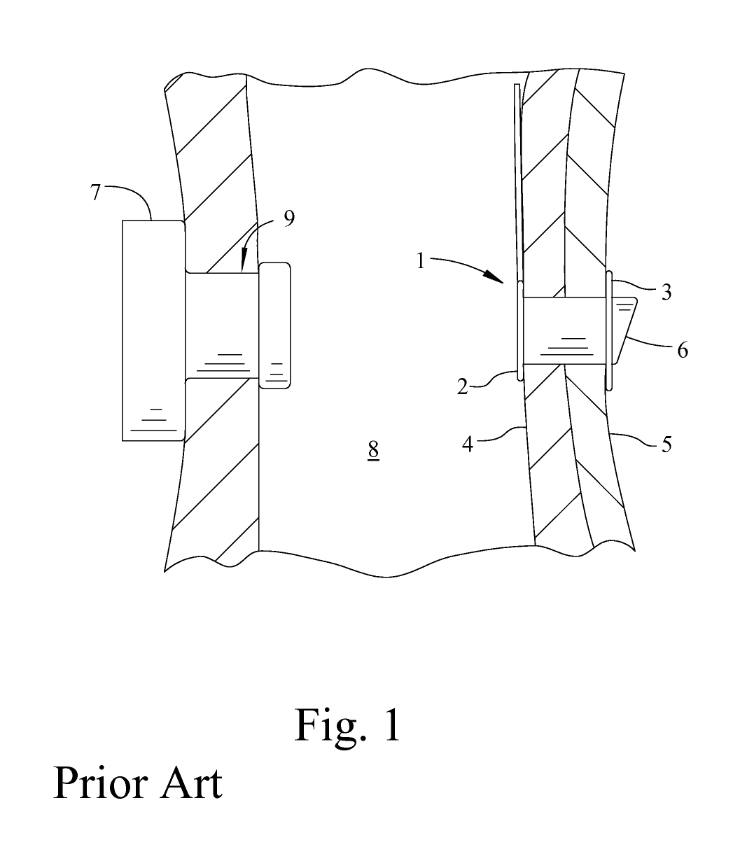

Referring now to FIG. 1, a voice prosthesis 1 of the prior art is shown in a cross-section of a users throat. One example of voice prosthesis 1 is sold by Inhealth Technologies having an address of 1110 Mark Avenue, Carpinteria, Calif. 93013, model number BE2022, and the details of this device are herein incorporated by reference. Voice prosthesis 1 includes flange seal 2 and flange seal 3 which are intended to provide a fluid seal against tracheal wall 4 and esophageal wall 5, respectively. Shroud 6 is most likely intended to deflect or route fluid and food passing through the esophagus around prosthesis 1 so that the internal check valve in prosthesis 1 does not become clogged with food. Also shown in FIG. 1 is tracheostoma button 7, installed in stoma 9 in a patient's throat wall through which breathing takes place. A shortcoming in the design of prosthesis 1 occurs when fluid and/or food in the esophagus penetrates seals 3 and 2 and enters into the tracheal passage or windpipe 8 region .

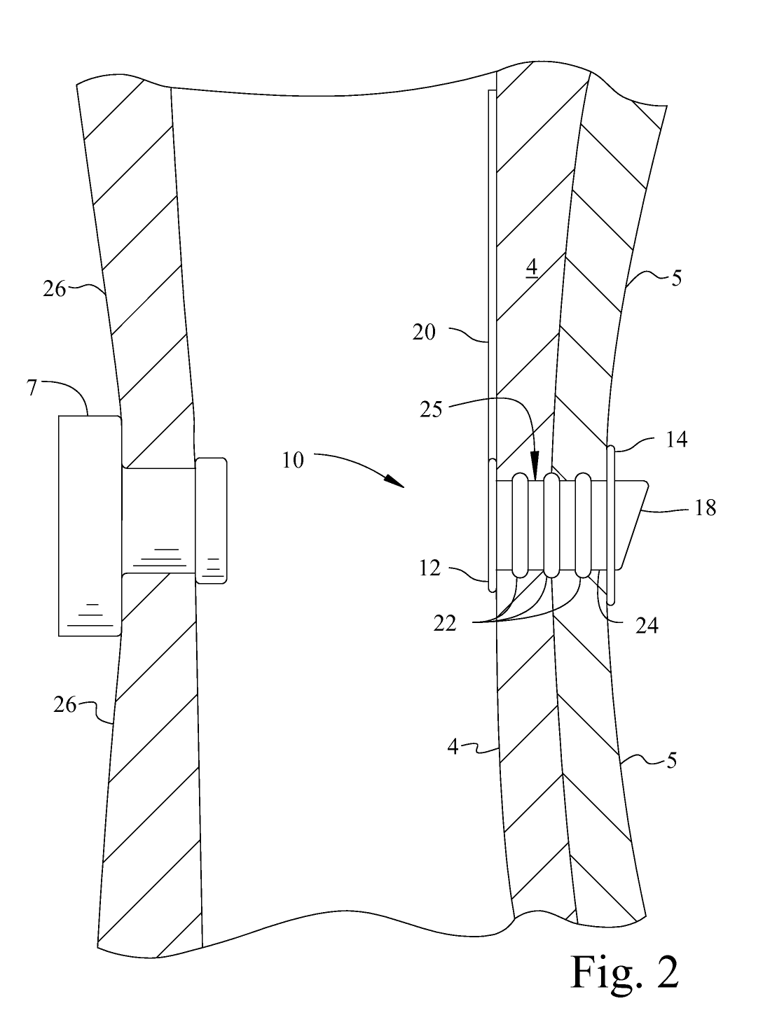

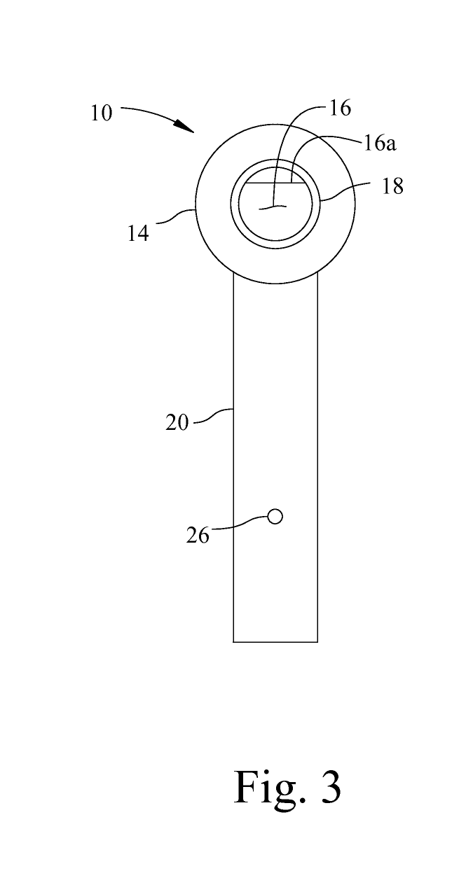

Referring now to FIGS. 2 and 3, a voice prosthesis 10 according to the present invention is shown. FIG. 2 is a cross-sectional side view of a patient's throat depicting prosthesis 10 situated in a tracheal-esophageal stoma 25 between tracheal wall 4 and esophageal wall 5, and FIG. 3 is a front view of prosthesis 10. Voice prosthesis 10 includes disk shaped retainer flanges 12 and 14, an internal check valve 16, hood or shroud 18 and installation strap 20. The improvement over the prior art in prosthesis 10 is my addition of annular rib or ridge seals 22 that are integrally formed in the cylindrical lateral surface 24 of prosthesis 10. The rounded protrusion of rib seals 22 provides an improved fluid seal when prosthesis 10 is installed in a patient's tracheal-esophageal stoma 25. It is also contemplated that the body tissues in contact with seals 22 and cylindrical lateral surface 24 will conform to the cross-sectional profile created by seals 22 and cylindrical lateral surface 24 to provide a more effective fluid seal for prosthesis 10 over the prior art. Strap 20 includes aperture 26 therethrough that engages an installation device (not shown) useful to medical personnel or users in placement of voice prosthesis device 10 into tracheal-esophageal stoma 25. Tracheostoma button 7 is also shown disposed in a stoma in external throat wall 26.

The typical length of prosthesis 10, the distance between retainer flanges 12 and 14 for the cylindrical portion 24 of voice prosthesis 10, varies from 6 to 28 millimeters and typical cylindrical diameters for surface 24 are between 16 and 20 FR. FR represents French scale where 1 FR=⅓ mm. The height above surface 24, width and spacing of annular seals 22 may be varied so that from patient to patient, the best fluid seal results are achieved. A typical height for rib seals 22 is 0.010 to 0.030 inches, though it may be necessary in certain circumstances to vary the height dimension outside this range based upon the application of prosthesis 10 to a particular patient. Separation spacing between rib seals 22 may be varied to achieve optimal results. Although three rib seals 22 are shown in FIGS. 2 and 3, it is contemplated that as few as a single rib seal 22 is required to achieve an improved seal over a prior art voice prosthesis.

Voice prosthesis 10 is fabricated in a variety of lengths and cylindrical diameters to accommodate the varying dimensions of an individual patient's tracheal-esophageal stoma wherein prosthesis 10 is placed. Prosthesis 10 may be fabricated from a semi-rigid resilient and flexible silicone compound that is readily sterilized, though other suitable materials are contemplated. Any well known geometric contour is contemplated for the cross-sectional profile of seals 22, including but not limited to semi-circular or round, elliptical, hexagonal, or the like. Most importantly, seals 22 should have a cross-sectional profile that is rounded and non-irritating to the patient. Check valve 16 is a simple flap type valve that is integrally formed in device 10 and fabricated to pivot along a thin portion of flap 16 at 16a.

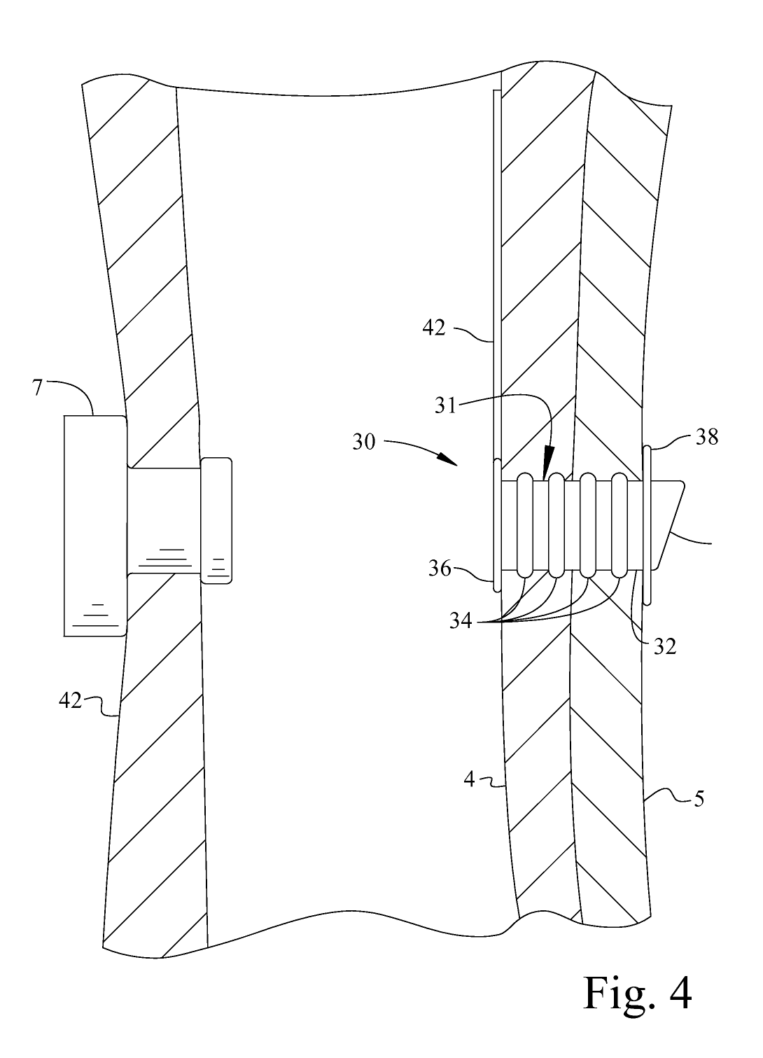

Referring now to FIG. 4, a cross-sectional side view of a patient's throat depicting another voice prosthesis 30 according to the present invention situated in a tracheal-esophageal stoma 31 between tracheal wall 4 and esophageal wall 5. Voice prosthesis 30 is nearly identical to prosthesis 10 except for the dimensional length of cylindrical portion 32 between retainer flanges 36 and 38. In this embodiment, four rib seals 34 are provided so that an improved fluid seal is achieved. Also shown in FIG. 4 are shroud 40 and installation strap 42. Other than the increased length of cylindrical portion 32, voice prosthesis 30 is identical in function and form to prosthesis 10 yet includes an additional rib seal 34 for the added length of cylindrical portion 32. A check valve (not shown) is situated within cylindrical portion 32 and is identical to check valve 16. Also shown is tracheostoma button 7 installed in the external wall 42 of the users throat.

The improvement over the prior art discussed herein is applicable to all voice prosthesis devices presently known in the art wherein a cylindrical body and disk like retainer flanges attached over the ends of the cylindrical body are used. The addition of rib seals to any prior art device will enhance the fluid seal thereof for users of voice prosthesis devices.

While the invention has been illustrated and described in detail in the drawings and foregoing description of the preferred embodiments, the same is to be considered as illustrative and not restrictive in character, it being understood that only the preferred embodiments have been shown and described and that all changes and modifications that come within the spirit of the invention are desired to be protected.

What I claim is:

1. A voice prosthesis device comprising:
a hollow cylindrical tube having an external lateral surface, a first open end, a second open end, and at least one annular protrusion extending radially outward a fixed distance from the external lateral surface of said hollow cylindrical tube, and wherein the radius of said tube is sized for insertion into a stoma connecting a users trachea and esophagus;
low pressure check valve means for allowing air to pass through said cylindrical tube when air pressure on one side of said cylindrical tube exceeds a predetermined level, said valve means being disposed within said cylindrical tube;
a first retainer flange attached over said first open end of said hollow cylindrical tube, said first retainer flange having a disk-like shape and an aperture aligned with said first open end; and
a second retainer flange attached over said second end of said hollow cylindrical tube, said second retainer flange having a disk-like shape and an aperture aligned with said second open end.

2. The device of claim 1 wherein said annular protrusion has a semi-circular cross-section at the distal end thereof.

3. The device of claim 1 wherein said annular protrusion has a rounded cross-section at the distal end thereof.

4. The device of claim 1 wherein said voice prosthesis device is fabricated from a semi-rigid resilient and flexible material.

5. A voice prosthesis device for insertion in a stoma between the trachea and the esophagus, said voice prosthesis device comprising:
a hollow cylindrical tube having a lateral surface, a first end, a second end and at least one annular rib extending radially outward a fixed distance from said lateral surface, and wherein said hollow cylindrical tube has a radius sized to correspond with the stoma between the trachea and the esophagus;
low pressure check valve means disposed within said tube for allowing air to pass through said tube when air pressure at the first end of said tube exceeds a predetermined level versus the air pressure at the second end of said tube;
a first retainer flange means disposed over the first end of said tube for retaining said cylindrical tube in position in the stoma; and
a second retainer flange means disposed over the second end of said tube for retaining said cylindrical tube in position in the stoma.

6. The device of claim 5 wherein said voice prosthesis device is fabricated from a semi-rigid flexible resilient material.

7. The device of claim 6 wherein said annular rib has a semi-circular cross-section at the distal end thereof.

8. A voice prosthesis device for insertion in a stoma between the trachea and the esophagus, said voice prosthesis device comprising:
a hollow cylindrical tube having a lateral surface, a first end, a second end and a first annular rib extending radially outward a predetermined distance from said lateral surface, and wherein said hollow cylindrical tube has a radius sized to correspond with the stoma between the trachea and the esophagus;
a low pressure check valve means disposed within said cylindrical tube for allowing air to pass through said tube when air pressure at the first end of said tube exceeds a predetermined level versus the air pressure at the second end of said tube;
a first retainer flange means disposed over the first end of said tube for maintaining said cylindrical tube in position in the stoma; and
a second retainer flange means disposed over the second end of said tube for maintaining said cylindrical tube in position in the stoma.

9. The device of claim 8 wherein said hollow cylindrical tube includes a second annular rib extending radially outward a predetermined distance from the lateral surface of said tube.

10. The device of claim 9 wherein said first and second annular ribs have a semi-circular cross-section at their distal tip.

11. The device of claim 10 wherein said device is fabricated from a semi-rigid flexible and resilient material.
